(19) United States
(12) Patent Application Publication
(10) Pub. No.: US 2017/0150985 A1
(43) Pub. Date: Jun. 1, 2017

(54) ULTRASONIC SURGERY APPARATUS, GRIPPING MEMBER PROVIDED ON JAW THEREOF, AND METHOD FOR PRODUCING GRIPPING MEMBERS

(71) Applicant: Ligasonix, Inc., New York,, NY (US)

(72) Inventor: Eung Kook KIM, Seocho-gu (KR)

(21) Appl. No.: 15/122,401

(22) PCT Filed: Feb. 28, 2014
(86) PCT No.: PCT/KR14/01708

§ 371 Date: Jan. 30, 2017

(51) Int. Cl. A61B 17/32; B29C 45/00

(52) U.S. Cl. CPC A61B 17/320092; B29C 45/0053; A61B 2017/2825

(57) ABSTRACT

Disclosed is a gripping member provided on a jaw which grips a surgical site while tilting along with an amputator in an ultrasonic surgical apparatus for amputating the surgical site, the gripping member comprising a body portion formed along the lengthwise-direction of the jaw and coupled thereto; and a fixing portion having a through-hole at one end through which a shaft penetrates, and extending from the body portion along the lengthwise-direction thereof toward the shaft provided so as to tilt the jaw, so that the body portion is not released from the jaw.

### TECHNICAL FIELD

The present invention relates to a surgical apparatus utilizing ultrasonic waves, and more particularly, the present invention relates to a gripping portion provided on a jaw, the gripping portion can be fixed stably without being separated by vibration of ultrasonic waves, and a surgical apparatus utilizing ultrasonic waves using the same.

### BACKGROUND ART

Recently, surgical apparatuses using ultrasonic waves are being developed. Among them, the surgical apparatuses similar with harmonic scalpel are the most representative.

The surgical apparatuses having such shape normally comprises: a gun-shaped handle unit gripped in the operator's hand; an amputator provided in front of the handle unit for amputating a surgical site using ultrasonic waves transmitted from a vibration unit provided in the handle unit; and a jaw provided at a position facing with the amputator for gripping a surgical site together with the amputator.

Here, the jaw which grips a surgical site together with an amputator is normally provided with a gripping portion made from a synthetic resin, so that the gripping portion and the amputator together grip a surgical site.

For producing a conventional gripping portion, an additional fixing portion a portion of which is inserted in one side of a jaw to be coupled with the jaw is used to fix the gripping portion with the jaw which requires an additional process.

The additional fixing portion and the additional manufacturing process for fixing the gripping portion and jaw together take time and cost. In addition, such construction may result in separation of the fixing portion as the vibration of the amputator is transmitted to the jaw, or even separation of the gripping portion from the jaw by the vibration.

Because a surgical apparatus utilizing ultrasonic waves is used inside of a patient's body, separation or detachment of the fixing portion or gripping portion can cause a severe problem.

### DETAILED DESCRIPTION OF THE INVENTION

### Technical Objects

An aspect of the present invention is to provide a gripping portion provided on a jaw of a surgical apparatus utilizing ultrasonic waves, the gripping portion can be fixed stably without being separated by vibration of ultrasonic waves, and a surgical apparatus utilizing ultrasonic waves using the same which can be manufactured at a lower cost due to simplified process.

The invention is not restricted to the technical objective set forth above. The above and other aspects of the invention not described herein will become apparent to those skilled in the art to which the invention pertains by referencing the detailed description of the invention below.

### Means for Achieving the Technical Object

The gripping portion provided on a jaw, which grips a surgical site together with an amputator by tilting from a surgical apparatus utilizing ultrasonic waves in the present embodiment in accordance with the present invention, comprises: a body portion formed along the lengthwise-direction of the jaw and coupled thereto; and a fixing portion having a through-hole at one end through which a shaft penetrates, and extending from the body portion along the lengthwise-direction thereof toward the shaft provided so as to tilt the jaw, so that the body portion is not released from the jaw.

The body portion can be formed comprising a first body portion having a length corresponding with the length of the jaw and a second body portion formed by protruding out from the underside of the first body portion and coupled in the coupling groove formed on the jaw.

To prevent a surgical site from being released from between the amputator and the jaw, the body portion can be provided with a structural pattem for further comprising a gripping portion on the top side of the first body portion, wherein the gripping portion is substantially formed in a plurality of grooves crisscrossing in the lengthwise and widthwise directions of the body portion.

In addition, the fixing portion is substantially formed extending from the second body portion in the lengthwise direction of the second body portion towards the shaft.

Here, the second body portion can be inserted into the coupling groove in a slidable way in the lengthwise direction, and the fixing portion is not coupled with the coupling groove.

The surgical apparatus utilizing ultrasonic waves in accordance with another aspect of the present invention comprises: a gun-shaped handle unit gripped in a hand of an operator; an amputator provided in front of the handle unit to amputate a surgical site utilizing the ultrasonic waves generated by a vibration unit provided inside of the handle unit; and a jaw provided at a position facing with the amputator, tiltable by an additional shaft to be attached to the amputator to grip a surgical site, and provided with a gripping portion formed on the surface attaching with the amputator to contact with the surgical site; and further comprising a body portion formed along the lengthwise direction of the jaw and coupled with the jaw, and a fixing portion formed by extending from the body portion towards the shaft for tilting the jaw along the lengthwise direction of the body portion and formed with a through hole where the shaft penetrates through.

Here, the body portion can be formed comprising a first body portion having a length corresponding with the length of the jaw and formed with a gripping portion which is formed in a plurality of grooves crisscrossing in the lengthwise and widthwise directions of the first body portion, and a second body portion formed by protruding out from the underside of the first body portion and coupled in the coupling groove formed on the jaw.

In addition, the fixing portion is formed by extending along the second body portion in the lengthwise direction towards the shaft, and the second body portion can be inserted into the coupling groove in a slidable way in the lengthwise direction, and the fixing portion is not coupled with the coupling groove.

The method for producing the gripping portion in accordance with another aspect of the present invention, which is a method for producing a body portion comprising a first body portion provided to a surgical apparatus utilizing ultrasonic waves for amputating a surgical site, whose one end is provided on the top side and coupled with a jaw which is tiltable by an additional shaft, to contact with and grip a surgical site together with an amputator, and a second body portion formed by extending from the underside of the first body portion and coupled with the jaw, and a gripping portion having a fixing portion formed by extending from the second body portion, wherein the method comprises: a first step for forming the body portion and the fixing portion in a long, linear shape by injection molding; a second step for cutting the body portion and the fixing portion injection molded in the first step into a predetermined length; a third step for forming the fixing portion from the body portion cut in the second step, by cutting off the upper portion of the fixing portion leaving the upper portion of the second body portion, and increasing the gap between the fixing portion and the first body portion by increasing the gap between the first body portion and the second body portion; and a fourth step for forming a through hole in the fixing portion formed in the third step in the widthwise direction.

Here, the second step can be characterized by cutting off the body portion leaving a length longer than that of the jaw.

In addition, the first step can be characterized by further comprising a step of forming a gripping portion on the top side of the first body portion in a form of a plurality of grooves crisscrossing in the lengthwise and widthwise directions of the first body portion.

In addition, the fixing portion can be characterized by being formed extending from the second body portion in the direction of the shaft and the first body portion is not formed on the top side.

### Effects of the Invention

The effects of the present invention devised to solve above problems are as follows.

Firstly, regarding the gripping portion provided on the jaw of the surgical apparatus utilizing ultrasonic waves, since a fixing portion extending from the body portion and fixed by the shaft is formed in an integral portion, the gripping portion can be fixed reliably without the worry of being detached from the jaw, even without any additional means for fixing.

Secondly, since the fixing portion is injection molded as an integral member of the body portion, the step of coupling the gripping portion and jaw using an extra fixing means can be eliminated, simplifying the manufacturing process.

The effects of the invention are not restricted to those set forth above. The above and other aspects of the invention will become apparent to those skilled in the art to which the invention pertains by the description of the claims.

### BRIEF DESCRIPTION OF THE DRAWINGS

FIG. 1 is a schematic diagram showing the construction of a conventional surgical apparatus utilizing ultrasonic waves,

FIG. 2 is a schematic diagram showing a jaw applied with a gripping portion for gripping a surgical site of the surgical apparatus utilizing ultrasonic waves of FIG. 1,

FIG. 3 is a schematic diagram showing a jaw applied with a gripping portion for gripping a surgical site of the surgical apparatus utilizing ultrasonic waves of in the present embodiment in accordance with the present invention.

FIG. 4 is an exploded perspective view of the gripping portion and jaw coupled together of the surgical apparatus utilizing ultrasonic waves of FIG. 2,

FIG. 5 is a schematic diagram showing the construction of the surgical site gripping portion of FIG. 2,

FIG. 6 is a side view of the surgical site gripping portion of FIG. 5,

FIG. 7 is a cross-sectional view of the surgical site gripping portion of FIG. 5,

FIG. 8 is a schematic diagram showing the surgical apparatus utilizing ultrasonic waves of FIG. 2 approaching a surgical site,

FIG. 9 is a schematic diagram showing the surgical apparatus utilizing ultrasonic waves of FIG. 8, gripping and amputating a surgical site,

FIG. 10 is a plan view showing the deformed shape of the gripping portion of FIG. 2,

FIG. 11 is a plan view showing another deformed shape of the gripping portion of FIG. 2,

FIG. 12 is a diagram showing the step 1 of the manufacturing process of the gripping portion of the surgical apparatus utilizing ultrasonic waves of FIG. 2,

FIG. 13 is a diagram showing the step 2 of the manufacturing process of the gripping portion of the surgical apparatus utilizing ultrasonic waves of FIG. 2,

FIG. 14 is a diagram showing the step 3 of the manufacturing process of the gripping portion of the surgical apparatus utilizing ultrasonic waves of FIG. 2, and

FIG. 16 is a diagram showing the step 4 of the manufacturing process of the gripping portion of the surgical apparatus utilizing ultrasonic waves of FIG. 2,

<Numbering Scheme of the Major Parts of the Drawings>

| | |
|---|---|
| 100: handle unit | 200: vibration unit |
| 300: transmission rod | 400: amputator |
| 500: rod cover | 600: jaw |
| 700: gripping portion | 710: body portion |
| 720: fixing portion | S: surgical site |

### MODE FOR CARRYING OUT THE INVENTION

The surgical apparatus utilizing ultrasonic waves, gripping portion provided on the jaw of the surgical apparatus utilizing ultrasonic waves, and method for producing the gripping portion are described in detail with preferable embodiments referring to accompanying drawings. It should be noted that the purpose of the description is not to limit the present invention to a specific form but to facilitate clear understanding with the embodiments.

In addition, in the description of the embodiments in accordance with the present invention, same elements shall be referred to with same names and numerals, without further description.

Meanwhile, in the description of the embodiments in accordance with the present invention, since an embodiment of a gripping portion provided on a surgical apparatus utilizing ultrasonic waves for amputating a surgical site can be included in an embodiment of the surgical apparatus utilizing ultrasonic waves, it will be included in the description of an embodiment of the surgical apparatus utilizing ultrasonic waves.

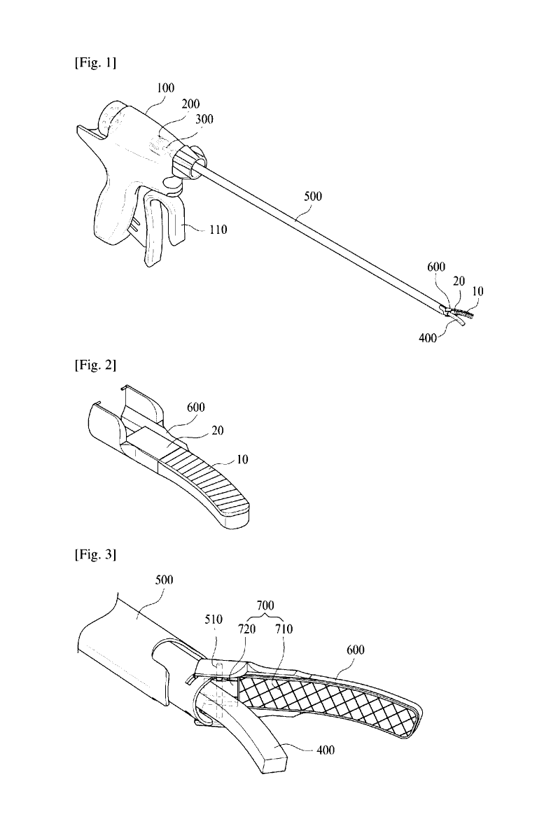

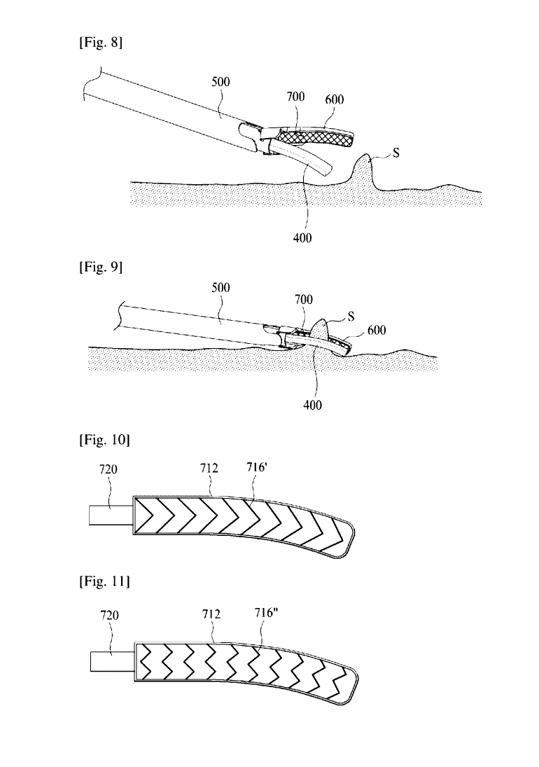

Referring to FIGS. 1 through 12, the configuration of the surgical apparatus utilizing ultrasonic waves in the present embodiment in accordance with the present invention is described below.

The surgical apparatus utilizing ultrasonic waves in the present embodiment in accordance with the present invention comprises: a handle unit 100 gripped by an operator, a vibration unit 200, a transmission rod 300, an amputator 400, a rod cover 500, a jaw 600 and a gripping portion 700.

The handle unit 100 is provided in a gun-shape for being held in a hand of an operator, comprising: a vibration unit 200 inside for generating vibration, a transmission rod 300 connected with the vibration unit 200 and a rod cover 500 connected in forward direction. In addition, an additional operating means 110 is provided to control the wavelength, amplitude and frequency of the ultrasonic waves generated by the vibration unit 200.

The vibration unit 200 generates ultrasonic waves by receiving electric signals from input terminals. (However, the electric circuit and wiring for transmitting the signals between the vibration unit 200 and the input terminals are not indicated in the drawings.)

The frequency of the ultrasonic waves generated by the vibration unit 200 varies by the conditions of the transmission rod 300 and amputator 400 which will be described later. That is, the ultrasonic waves generated by the vibration unit 200 can be adjusted by the configuration and material of the transmission rod 300 and/or design of the gain steps and operating length of the amputator 400.

For example, if the transmission rod 300 is made of titanium in a cylindrical shape, the gain step and operating length of the amputator 400 is about 20 mm and 15 mm, respectively, the vibration unit 200 will generated ultrasonic waves at about 55.5 kHz of frequency.

In addition, the wavelength, amplitude and frequency of the ultrasonic waves generated by the vibration unit 200 can be controlled with the operating means 110 taking the surgical site S to be amputated into consideration.

For example, if the surgical site S is a thick tissue or containing a blood vessel, the intensity of the ultrasonic waves should be adjusted to be higher, or the intensity can be controlled to be lower to avoid hindrance of the byproducts of amputation to the operation.

Meanwhile, the transmission rod 300 connects the vibration unit 200 and amputator 400 to transmit the ultrasonic waves generated by the vibration unit 200 to the amputator 400.

The transmission rod 300 in the present embodiment in accordance with the present invention is provided in a long, rod-shape whose one end and the opposite end are connected to the vibration unit 200 described earlier and amputator 400 described later, respectively.

While not shown in the drawings, the transmission rod 300 can be configured in a long, thin, cylindrical bar so that the amputator 400 can be inserted in a hole formed on a trocar used in laparoscopy to reach a surgical site S easily.

In addition, the length of the transmission rod 300 can be provided sufficiently for laparoscopy, and allow for the ultrasonic waves generated by the vibration unit 200 for repetition of the vibration nodes (the points where the intensity of ultrasonic wave is substantially zero) and vibration anti-node (a concept opposite to the vibration node, the points where the intensity of ultrasonic wave is the highest) by a plurality of number lengths.

In addition, while the transmission rod 300 can be made with various materials, titanium material would be preferable for efficient transmission of the 55.5 kHz of ultrasonic waves generated in the vibration unit 200 as described earlier.

In the present embodiment, the transmission rod 300 is formed in a long cylindrical bar shape, a portion of which is connected inside of the handle unit 100 and the opposite portion protrudes out forward.

The amputator 400 for cutting a surgical site S utilizing ultrasonic waves is provided on one end of the transmission rod 300 to receive the ultrasonic waves generated by the vibration unit 200 to cut a surgical site S.

More particularly, the amputator 400 in accordance with the present invention can be formed by being extended from one end of the transmission rod 300 described earlier, and can cut a surgical site S by pressing and gripping the surgical site S together with the jaw 600, to be described later, provided on the opposite side, using vibration energy and thermal energy.

Here, the amputator 400 and the transmission rod 300 can be made of different materials in order to avoid the heat transfer from the amputator 400 to the transmission rod 300.

Since the amputator 400 operates utilizing ultrasonic waves, it does not have a sharp edge, different from other ordinary cutting devices. That is, the amputator 400 is designed to cut a surgical site S using vibration energy and thermal energy, thus, it is effective when amputating a surgical site S through which a blood vessel or vessels pass.

The amputator 400 in the present embodiment in accordance with the present invention can be formed with a smaller diameter than that of the transmission rod 300 and may be curved along in the lengthwise direction, as shown in the drawings. This is to facilitate stable contact or grip with a surgical site S, however, the shape can be changed to be fit for specific application. Here, the amputator 400 shall be so formed as to be able to make use of the vibration energy and thermal energy of the ultrasonic waves received from the vibration unit 200.

Since the amputator 400 should be able to amputate a surgical site S utilizing the ultrasonic waves transmitted through the transmission rod 300, it should have a gain step (indicating that the actual ratio of amplification is 1 or higher).

For a means to provide the amputator 400 with a gain step, the shape and structure of the amputator 400 can be diversified, In the present embodiment in accordance with the present invention, the lateral cross-sectional area of the amputator 400 is reduced to be smaller than that at the vibration node so that the energy of the ultrasonic waves can be amplified according to the ratio of the cross-sectional area. Accordingly, when the amputator 400 is formed by being extended from the transmission rod 300, a portion of the round cross-sectional area is flattened to reduce the area and to form a gain step.

The rod cover 500 surrounds the transmission rod 300 to protect the ultrasonic waves transmitted through the transmission rod 300. As such, the rod cover 500 in the present embodiment in accordance with the present invention is a long, hollow rod, allowing the transmission rod 300 to penetrate through the hollow portion.

In addition, the opposite end is coupled and fixed with the handle unit 100 described earlier, and the amputator 400 penetrates and exposed outwards through the one end.

The rod cover 500 in the present embodiment in accordance with the present invention has a length corresponding with the length of the transmission rod 300 described earlier, the opposite end is coupled with the handle unit 100 and the amputator 400 protrudes out from the hollow of the rod cover.

While the rod cover 500 is preferably not contacting with the transmission rod 300, however, it may be necessary to be connected if the transmission rod 300 is long. Here, the connection point is preferably be made on a plurality of the vibration nodes on the transmission rod 300 formed by the ultrasonic waves.

This is because, since the intensity of the ultrasonic waves is not zero at any other points than the vibration nodes, if the transmission rod 300 and rod cover 500 are connected at any other points than the vibration nodes, the energy of the ultrasonic waves being transmitted through the transmission rod 300 is transmitted to the rod cover 500 and lost (wasted).

That is, though not shown, the rod cover 500 and transmission rod 300 in the present embodiment in accordance with the present invention can be configured to contact with each other partially along on the lengthwise direction.

The jaw 600, positioned facing with the amputator 400 to grip a surgical site S by adhering to the amputator 400. As shown in the drawings, it is coupled to the opposite end of the rod cover 500 in a tiltable way using an additional shaft 510.

More particularly, the jaw 600, formed in a long shape having a certain length, whose one end is so configured as to allow tilting from the rod cover 500, enabling adjustment of the rotating angle. As such, the jaw 600 can grip the surgical site S together with the amputator 400 penetrating through and protruding out from the rod cover 500.

The jaw 600 in the present embodiment in accordance with the present invention is provided with a gripping portion 700 to be described later on one side. The gripping portion 700 is provided on one side of the jaw 600 to grip the surgical site S together with the amputator 400.

The jaw 600 coupled with the rod cover 500 in a tiltable manner with a tilting angle controllable by the operating means 110 described earlier. Accordingly, the gripping portion 700 and amputator 400 can grip the surgical site S.

That is, the jaw 600 is configured not to contacting directly with the surgical site S, but the gripping portion 700 provided on one side of the jaw grips the surgical site S together with the amputator 400.

When the surgical site S is gripped by the gripping portion 700 and amputator 400, the amputator 400 can amputate the surgical site S utilizing the ultrasonic waves generated by the vibration unit 200.

The jaw 600 in the present embodiment in accordance with the present invention can be curved along the lengthwise direction, as shown in the drawing, for stable gripping of the surgical site S. However, its actual shape can be formed selectively according to the surgical site S or application.

In addition, the jaw 600 in the present embodiment in accordance with the present invention is formed with a coupling groove 610 in which a portion of the gripping portion 700 is coupled with. As shown in the drawing, the coupling groove 610 is formed along the lengthwise direction of the jaw 600, and a portion of the gripping portion 700 is inserted in the coupling groove 610 to support the gripping portion 700 not to be detached from one side of the jaw 600.

Meanwhile, the gripping portion 700 provided on one side of the jaw 600 described earlier grips the surgical site S together with the amputator 400, at the position adjusted selectively according to the tilting condition of the jaw 600.

More particularly, the gripping portion 700 in the present embodiment in accordance with the present invention can be made of a synthetic resin in a form corresponding with that of the jaw 600 and provided on one side of the jaw 600.

The gripping portion 700 in the present embodiment in accordance with the present invention can comprise a body portion 710 and a fixing portion 720.

Here, the body portion 710 which is formed along the lengthwise direction of the jaw 600 is so configured that at least a portion of it is coupled with the jaw 600.

More particularly, the body portion 710 in the present embodiment in accordance with the present invention has a protruding portion which is inserted in the coupling groove 610 described earlier to facilitate stable coupling of the jaw 600 and gripping portion 700.

The body portion 710 in the present embodiment in accordance with the present invention can comprise: a first body portion formed with a length corresponding with that of the jaw 600; and a second body portion 714 protruding outwards from the bottom of the first body portion 712 and coupled with the coupling groove 610 formed on the jaw 600. The body portion 710 can, but not limited to, be formed as an integral member, instead of comprising the first body portion 712 and the second body portion 714.

The first body portion 712 in the present embodiment in accordance with the present invention whose length corresponds with that of the jaw 600 is provided with an additional gripping portion 716 on one side to grip a surgical site S together with the amputator 400.

Here, the first body portion 712 has an area corresponding with the size of the jaw 600, as described earlier, and the second body portion 714 is formed by protruding out from the bottom side.

More particularly, the first body portion 712 in the present embodiment in accordance with the present invention can be formed with an additional structural pattern and a gripping portion 716 on the top side of the first body portion 712 to prevent the surgical site S from being released from between the amputator 400 and jaw 600 when the surgical site S grips or cuts.

The gripping portion 716 is formed in a plurality of grooves crisscrossing in the lengthwise and widthwise directions of the first body portion 712 to prevent slip in the contacting surface with the surgical site S for firm gripping.

In addition, the gripping portion 716 prevents the surgical site S from slipping from the amputator 400 which is vibrating by ultrasonic waves.

While the gripping portion 716 in the present embodiment in accordance with the present invention is formed with a structural patter comprising a plurality of grooves crisscrossing in the lengthwise and widthwise directions of the first body portion 712, however, it may be formed in various other pattems.

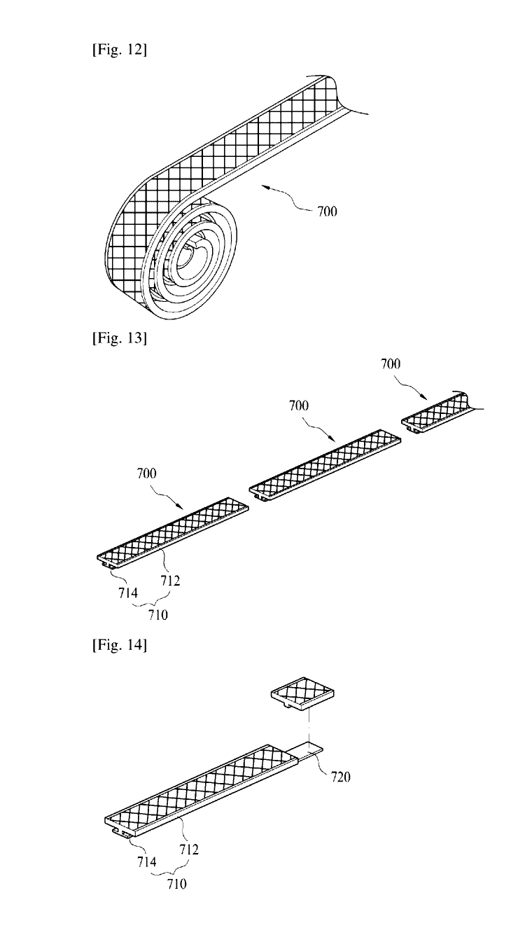

More particularly, the gripping portion 716 can be formed in a different shape than the simple perpendicularly crossing lines as shown in FIGS. 11 and 12, e.g., the examples of variants. The structural pattern of the gripping portion 716, as shown in FIGS. 11 and 12 can be replaced with angled, zigzag lines (716′, 716″) directing from the center to the opposite sides of the gripping portion 700.

As described above, the first body portion 712 is provided on the top side of the jaw 600 described earlier and formed with the gripping portion 716 for stable gripping of a surgical site S when gripping the surgical site S together with the amputator 400.

The second body portion 714, as shown in FIG. 7, protrudes from the bottom side of the first body portion 712 in an ‘H’ shape.

Here, the second body portion 714, as shown in FIG. 6, has relatively smaller width (A2) and area than the width (A1) of the first body portion 712, and a portion of the cross-section of the lower portion is sunk.

As the second body portion 714 is formed as described above, a portion of the second body portion 714 is coupled with by sliding into the coupling groove 610 formed on the jaw 600. As a result, the body portion 710 can be fixed on one side of the jaw 600.

More particularly, the second body portion 714 is formed on the first body portion 712 protruding from the opposite side to the side where the gripping portion 716 is formed, and inserted into the coupling groove 610 to fasten the first body portion 712 and jaw 600 without being released from mating member.

In the present embodiment in accordance with the present invention, a portion of the second body portion 714 is inserted into the coupling groove 610 along the lengthwise direction in a slidable manner.

This configuration of the body portion 710 provided on one side of the jaw 600 enables stable gripping and amputating a surgical site S together with the amputator 400.

Meanwhile, the fixing portion 720 is formed by extending from the body portion 710 towards the shaft 510 along the lengthwise direction of the body portion 710 to prevent the body portion 710 from separating from the jaw 600 and provided with a through hole 724 on one end through which the shaft 510 penetrates.

More particularly, the fixing portion 720 in accordance with an embodiment of the present invention protrudes from the first body portion 712 or second body portion 714 towards the shaft 510 and formed with a through hole 724 perpendicular with the lengthwise direction.

The extending fixing portion 720 is penetrated by the shaft 510 to provide a secondary fixture to prevent the body portion 710 from separating from the jaw 600.

The fixing portion 720 in the present embodiment in accordance with the present invention, as shown in FIGS. 5 and 6, is formed by extending from the second body portion 714 towards the shaft 510 and provided a through hole 724 on one side of the extension. That is, the fixing portion 720 is formed by an extension of the second body portion 714 and not provided with a first body portion 712 on the top side.

Accordingly, the fixing portion 720 is formed with the same width as of the second body portion 714.

Meanwhile, the fixing portion 720 is formed providing a gap between the first body portion 712 and second body portion 714, as shown in FIGS. 5 and 6, in order to provide the fixing portion 720 with more degree of freedom.

The purpose of providing the fixing portion 720 with degree of freedom is for further reinforcement of the performance of the fixing portion 720.

That is, if the fixing portion 720 has no degree of freedom the fixing portion 720 cannot buff vibration transmitted to it, and the fixing portion 720 may fail to be fixed, for example, by an unexpected external force caused by vibration applied to a point between the fixing portion 720 and the member fixed by the fixing portion 720.

As suc, the fixing portion 720 is preferably formed providing a gap between the first body portion 712 and second body portion 714, as shown in FIGS. 5 and 6, in order to provide the fixing portion 720 with more degree of freedom.

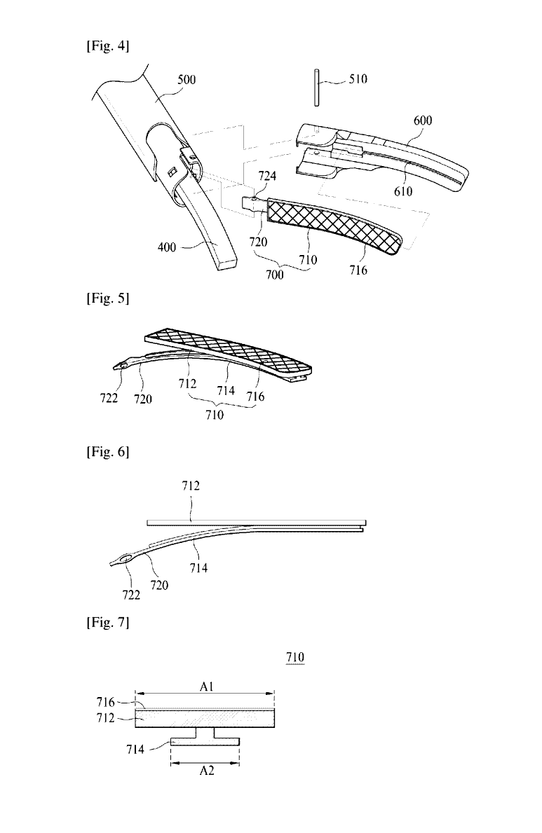

As the gripping portion 700 is so configured, considering the joint between the fixing portion 700 and the jaw 600, as shown in FIG. 4, the second body portion 714 is sliding-coupled through the coupling groove 610 formed on the jaw 600 in the first step.

Here, since the coupling groove 610 is formed in a long shape along the longwise direction of the jaw 600 and the second body portion 714 is also formed in a long shape corresponding with the shape of the groove with an ‘H-shaped’ cross-section, the second body portion 714 can be coupled with the coupling groove 610 by sliding.

Then, with the second body portion 714 coupled with the coupling groove 610, the through hole 724 formed in the fixing portion 720 is fastened by being penetrated by the shaft 510.

That is, the shaft 510 couples the jaw 600 and the rod cover 500 in a tiltable manner and penetrates the through hole 724 for coupling additionally.

Accordingly, even though the gripping portion 700 slide-couples with the jaw 600, it can maintain the coupled state stably without separating from the jaw 600 because the fixing portion 720 is fixed by the shaft 510.

When the amputator 400 vibrated by ultrasonic waves, the vibration is transmitted to the jaw 600 and separation of the gripping portion 700 can be prevented by fine vibration.

Since the conventional gripping portion 10 has not been provided with the fixing portion 720, the gripping portion 10 which is slide-coupled was fixed using an additional fixing member 20 as shown in FIG. 2 in order to prevent separation of the gripping portion 10 and jaw 600 by the vibration of the amputator 400.

Here, the conventional fixing portion 20 made a metallic material is coupled to the jaw 600 after coupling the gripping portion 10 with the jaw 600.

As described above, the conventional configuration requires an additional fixing portion 20 and the related steps in the manufacturing process. In addition, the conventional surgical apparatus utilizing ultrasonic waves has the problem that the conventional gripping portion 10 may be detached by the separation of the fixing portion 20.

To this end, the gripping portion 700 in accordance with the present invention can be formed as an integral member without additional fixing portion 20, simplifying the manufacturing process and substantially preventing separation of the gripping portion 700.

As described above, the gripping portion 700 in accordance with an embodiment of the present invention can maintain coupling state with the jaw 600 stably.

The surgical apparatus utilizing ultrasonic waves and the gripping portion 700 provided on the jaw 600 of the surgical apparatus utilizing ultrasonic waves are described in detail hereinabove. The gripping of the surgical site S by the surgical apparatus utilizing ultrasonic waves in accordance with an embodiment of the present invention is described in detail below.

As shown in FIG. 7, the gripping portion 700 and amputator 400 in accordance with an embodiment of the present invention approach dose to a surgical site S without contacting with each other and with the jaw 600 being tilted. Here, a portion of the surgical apparatus utilizing ultrasonic waves has already been inserted in the body of an operatee, and the surgical site S refers to a portion of the body which is the target of the operation.

After inserted into the body of the operate, the amputator 400 and jaw 600 grip the surgical site S or part to be cut off, as shown in FIG. 9. At this time, the operator tilts the jaw 600 by using the operating means 110, so that the amputator 400 and gripping portion 700 contact with and grip the surgical site S.

Then, the operator starts the vibration unit 200 to amputate the surgical site S.

At this time, the gripping portion 700 in accordance with an embodiment of the present invention is fixed is fixed to the jaw 600 doubly by the fixing portion 20, thus, can grip the surgical site S stably, not being detached from the jaw 600 by the vibration caused by ultrasonic waves.

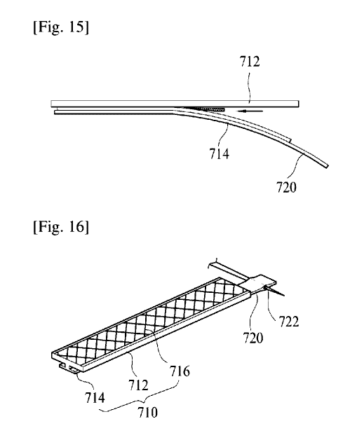

Referring to FIGS. 12 through 16, the process of manufacturing the gripping portion 700 of the surgical apparatus utilizing ultrasonic waves in accordance with an embodiment of the present invention is described below.

The manufacturing process of the gripping portion 700 in accordance with the present invention comprises a first step of injection molding the body portion 710 and fixing portion 720 combined in a long single part.

Here, the body portion 710 and fixing portion 720 may be injection molded with an identical material in a long, rod-like shape. The body portion 710 is injection molded with a cross-section area corresponding with the shape of the first body portion 712 and second body portion 714.

On the other hand, the body portion 710 and fixing portion 720 can be injection molded in addition with the gripping portion 716 described earlier on the portion which will become the first body portion 712.

That is, the body portion 710 and fixing portion 720 can be injection molded as a single part, or alternatively, the gripping portion 716 can be additionally formed on the top side of the first body portion 712. In addition, the body portion 710 and fixing portion 720 can be injection molded as a single part, and then formed with the gripping portion 716 additionally on the top side of the first body portion 712.

Above two processes can be selected by the manufacturer according to the situation.

The body portion 710 and fixing portion 720 injection molded in a long, single part in the step 1 is cut into unit members by a predetermined lengths in step 2. Here, since each of the cut units are processed into one gripping portion 700, they are cut by a length longer than that of the jaw 600.

The cut body portion 710 and fixing portion 720 have H-shape cross-sections and lengths longer than that of the jaw 600.

To form fixing portion 720, from each of the body portion 710 and fixing portion 720 cut in the step 2, the portion on top side of the fixing portion 720 is cut off, leaving the portion on the top side of the second body portion 714, of the first body portion 712, and the gap between the fixing portion 720 and first body portion 712 is increased by widening the gap between the first body portion 712 [symbol] second body portion 714 close to the fixing portion 720, in step 3.

That is, in the step 3, the body portion 710 and fixing portion 720 cut in the step 2 maintains only the first body portion 712 and second body portion 714. Then, one end in the lengthwise direction of the first body portion 712 is cut off so that a portion of the second body portion 714 becomes the fixing portion 720. Here, the gap between the fixing portion 720 and first body portion 712 can be increased to increase the degree of freedom to improve the functionality of the fixing portion 720.

Lastly, in step 4, a through hole 724 is formed in the fixing portion 720 formed in step 3 in the direction perpendicular with the lengthwise direction of the fixing portion 720. The method for forming the through hole 724 is not limited as long as the method can form the hole.

The gripping portion 700 in accordance with an embodiment of the present invention can be produced by the method described above. Each gripping portion 700 so produced is attached to each of the jaw 600 of a surgical apparatus utilizing ultrasonic waves.

From the foregoing it is believed that those skilled in the pertinent art will recognize the meritorious advancement of this invention and will readily understand that while the present invention has been described in association with a preferred embodiment thereof, and in the accompanying drawings, numerous changes and modifications may be made therein by those skilled in the art without departing from the spirit and scope of this invention. However, the present invention is not limited to the embodiments provided here but can be implemented in various ways within the scope of the claim.

What is claimed is:

1. A gripping portion provided on a jaw of a surgical apparatus utilizing ultrasonic waves for gripping a surgical site, comprising:
a body portion formed along the lengthwise direction of the jaw and attached to the jaw; and a fixing portion formed by extending from the body portion towards the shaft for tilting the jaw along the lengthwise direction of the body portion and formed with a through hole where the shaft penetrates through.

2. The gripping portion of claim 1,
wherein the body portion comprises: a first body portion having a length corresponding with the length of the jaw; and a second body portion formed by protruding out from the bottom side of the first body portion and coupled in the coupling groove formed on the jaw.

3. The gripping portion of claim 2,
wherein the body portion further comprises a gripping portion formed on the top side of the first body portion and provided with structural pattem to prevent the surgical site from being released from between an amputator and the jaw when amputating the surgical site, and
the gripping portion is characterized by being formed with a plurality of crisscrossing grooves.

4. The gripping portion of claim 2,
wherein the fixing portion is characterized by being formed extending from the second body portion in the lengthwise direction of the second body portion towards a shaft.

5. The gripping portion of claim 3,
wherein the second body portion is inserted into the coupling groove in a slidable way in the lengthwise direction, and the fixing portion is not coupled with the coupling groove.

6. A surgical apparatus utilizing ultrasonic waves comprising: a gun-shaped handle unit gripped by a hand of an operator; an amputator provided in front of the handle unit to amputate a surgical site utilizing the ultrasonic waves generated by a vibration unit provided inside of the handle unit; and a jaw provided at a position facing with the amputator, tiltable by an additional shaft to be attached to the amputator to grip a surgical site, and provided with a gripping portion formed on the surface attaching with the amputator to contact with the surgical site; and further comprising a body portion formed along the lengthwise direction of the jaw and coupled with the jaw, and a fixing portion formed by extending from the body portion towards the shaft for tilting the jaw along the lengthwise direction of the body portion and formed with a through hole where the shaft penetrates through.

7. The surgical apparatus utilizing ultrasonic waves of claim 6,
wherein the body portion comprises: a first body portion having a length corresponding with the length of the jaw and formed with a plurality of crisscrossing grooves on the top side; and a second body portion formed by protruding out from the bottom side of the first body portion and coupled in the coupling groove formed on the jaw.

8. The surgical apparatus utilizing ultrasonic waves of claim 7,
wherein the fixing portion is formed by extending along the second body portion in the lengthwise direction towards the shaft, and the second body portion can be inserted into the coupling groove in a slidable way in the lengthwise direction, and the fixing portion is not coupled with the coupling groove.

9. A method for producing a gripping portion used in a surgical apparatus utilizing ultrasonic waves for amputating a surgical site, the gripping portion provided on a jaw whose one end can be tilted by a shaft, gripping a surgical site together with an amputator, and the gripping portion comprises: a body portion comprising a first body portion provided on the top side of the jaw and a second body portion formed by protruding out from the bottom side of the first body portion and coupled with the jaw; and a fixing portion formed by extending from the second body portion, the method comprising:
a first step for injection-molding the body portion and the fixing portion in a long, single part;
a second step for cutting the body portion and the fixing portion injection-molded in the first step into predetermined lengths;
a third step for cutting off the upper portion of the fixing portion while leaving the upper portion of the second body portion, of the first body portion, in order to be able to form the fixing portion on the body portion cut in the second step, and increasing the gap between the fixing portion from the first body portion by increasing the gap between the first body portion and the second body portion adjacent to the fixing portion; and a fourth step for forming a through hole in the fixing portion formed in the third step in the direction perpendicular with the lengthwise direction.

10. The method of claim 9,
wherein the second step is characterized by,
the body portion is cut off with a length longer than the length of the jaw.

11. The method of claim 9,
wherein the first step is characterized by,
further comprising a step of forming a gripping portion on the top side of the first body portion in a form of a plurality of grooves crisscrossing in the lengthwise and widthwise directions of the first body portion.

12. The method of claim 9,
wherein the fixing portion is characterized by,
being formed extending from the second body portion in the direction of the shaft and the first body portion is not formed on the top side.
